(19) United States
(12) Patent Application Publication
(10) Pub. No.: US 2026/0240470 A1
(43) Pub. Date: Aug. 20, 2026

(54) SYSTEM AND METHOD FOR EMOTIONAL STATE MONITORING AND REGULATION

(71) Applicant: VERIGLOW INC, SMYRNA, GA (US)

(72) Inventors: Thomas Guy, III, Smyrna, GA (US); Khalil I. Carter, Smyrna, GA (US); Caron G. Dorvil, Smyrna, GA (US); Stevens Rentus, Smyrna, GA (US)

(73) Assignee: VERIGLOW INC, SMYRNA, GA (US)

(21) Appl. No.: 19/541,194

(22) Filed: Feb. 16, 2026

Related U.S. Application Data
(60) Provisional application No. 63/759,444, filed on Feb. 17, 2025.

(51) Int. Cl. A61B 5/16; A61B 5/00; A61M 21/00; A61M 21/02

(52) U.S. Cl. CPC A61B 5/165; A61B 5/0002; A61B 5/4836; A61B 5/7207; A61B 5/7264; A61B 5/7275; A61B 5/742; A61M 21/02; A61B 2560/0443; A61M 2021/0022; A61M 2021/0027; A61M 2021/0044; A61M 2209/088

(57) ABSTRACT

A system and method for monitoring and regulating emotional states, implemented via a computer system, is disclosed. A wearable module continuously acquires various physiological parameters, including dermal temperature, electrodermal activity, cardiovascular waveforms, and motion data. This data is processed to generate an emotional state indicator. The system provides biofeedback to the user through variable chromatic illumination and auditory cues corresponding to the detected emotional state. Intervention protocols are initiated when predetermined emotional thresholds are exceeded and the efficacy of these interventions are evaluated and modified based on performance metrics. In one embodiment tailored for educational environments, a central monitoring system aggregates emotional data from multiple wearable modules. This provides teachers with real-time individual and aggregated emotional states and recommending classroom activities for emotional regulation based on the aggregated data.

### RELATED APPLICATIONS

This application claims priority to U.S. Provisional Patent Application Ser. No. 63/759,444, filed Feb. 17, 2025, now pending.

### FIELD OF THE INVENTION

The present invention relates to a system and method for real-time emotional state monitoring and regulation wherein wearable technology is utilized to acquire a variety of physiological parameters in educational and other environments. More particularly, the method integrates a central monitoring system featuring a display interface and a recommendation module to aggregate and analyze emotional data from multiple users. Therefore, actionable insights are provided for teachers and peer-to-peer connection support between users experiencing similar emotional states. The method further employs an artificial intelligence (AI) processor to adapt intervention protocols and enhance user engagement through biofeedback mechanisms including variable chromatic illumination, auditory cues, and gamification elements.

### BACKGROUND OF THE INVENTION

The present invention provides an innovative system and method for real-time emotional state monitoring and regulation. The system utilizes advanced wearable technology to capture specific physiological parameters such as dermal temperature, electrodermal activity, cardiovascular waveforms, and motion data within educational and therapeutic environments. The system integrates a robust central monitoring system featuring a display interface with class, individual, and temporal view modes. This allows educators to aggregate and analyze real-time emotional data. This holistic approach provides users with immediate insights into their emotional states thereby facilitating timely interventions to improve mental well-being and fostering a supportive atmosphere through built-in biofeedback mechanisms like variable chromatic illumination, auditory cues, and peer-to-peer connection functionalities.

The user-centric design of the wearable modules can be adapted for wear as a personal adornment such as a ring, bracelet, necklace, earring or the same through modular attachment mechanisms. The physical docking interface for automated charging and data extraction allows for seamless integration of emotional data tracking and feedback. To enhance emotional awareness and regulation by addressing the growing need for mental health resources, the system employs machine learning algorithms and artificial intelligence units to adapt intervention protocols based on historical efficacy metrics and incorporates gamification elements to promote consistent use and progress tracking for self-awareness goals. The method is further characterized by customizable emotional thresholds for triggering interventions and incorporates features like a digital journal for storing temporal emotional state data and user reflections. This contributes to a comprehensive solution aimed at enhancing emotional resilience and overall educational experiences.

### SUMMARY

The present invention provides a method for real-time emotional state monitoring and regulation by utilizing wearable technology to assess a variety of physiological parameters in educational and other environments. In one embodiment, the method involves acquiring physiological parameters, such as dermal temperature, electrodermal activity, cardiovascular waveforms, and motion data, through a wearable module. These parameters are processed to generate an emotional state indicator which is conveyed through biofeedback mechanisms including variable chromatic illumination and auditory cues. The system analyzes emotional state patterns, initiates intervention protocols when predetermined emotional thresholds are exceeded, and evaluates the efficacy of these interventions using a comparative analysis of pre- and post-intervention indicators.

The apparatus includes a plurality of wearable modules equipped with sensor arrays, processors for real-time analysis, and a central monitoring system with a data aggregation processor that compiles data from multiple modules. This system provides visual representations of emotional states via a display interface and a recommendation module configured to suggest classroom activities based on aggregated emotional data.

The system further incorporates machine learning algorithms and artificial intelligence units for adaptive interventions based on historical efficacy data, geolocation components for peer-to-peer connections between users experiencing similar emotional states, and gamification elements to encourage emotional self-awareness and consistent use through achievement metrics and rewards. Overall, this disclosure aims to enhance emotional regulation and monitoring particularly in educational settings.

Unless otherwise defined, all technical and scientific terms used herein have the same meaning as commonly understood by one of ordinary skill in the art. Although methods and materials similar or equivalent to those described herein can be used in the practice or testing of any described embodiment, suitable methods and materials are described below. In addition, the materials, methods, and examples are illustrative only and not intended to be limiting.

In case of conflict with terms used in the art, the present specification, including definitions, will control. The foregoing summary is illustrative only and is not intended to be in any way limiting. In addition to the illustrative aspects, embodiments, and features described above, further aspects, embodiments, and features will become apparent by reference to the drawings and the following detailed description and claims.

### BRIEF DESCRIPTION OF THE DRAWINGS

The present embodiments are illustrated by way of the figures of the accompanying drawings, which may not necessarily be to scale, in which like references indicate similar elements, and in which:

FIG. 1 is a block diagram illustrating the system architecture and workflow for the emotional state monitoring and regulation method as provided herein;

FIG. 2 is a flowchart detailing the step-by-step process of data acquisition, analysis, intervention, and efficacy evaluation;

FIG. 2A represents the method for emotional state monitoring and regulation as provided herein;

FIG. 2B provides a detailed operational description of the central monitoring system functions as provided herein;

FIG. 2C is a block diagram illustrating the system architecture and data flow for the emotional state monitoring and regulation system as provided herein;

FIG. 3 is a functional block diagram illustrating the data flow between the wearable module and the central monitoring system;

FIG. 4 is a flowchart showing the emotional monitoring and regulation processes;

FIG. 5 is a flowchart showing the process of data aggregation from the wearable modules;

FIGS. 6, 7, and 8 are illustrations showing the wearable module configured as personal adornments further demonstrating its modular attachment mechanism;

FIG. 9 is an illustration depicting the central monitoring system implemented within a classroom environment, showing a teacher interface displaying aggregated emotional states;

FIGS. 10, 11, and 12 depict perspective views of docking interfaces for individual wearable modules or a plurality of wearable modules as provided herein;

FIG. 13 features a perspective view of a single detachable wearable module;

FIG. 14 is an illustration showing a perspective view showing the detachability of the module as provided herein;

FIG. 15 is a front elevation view of the wearable device shown in FIG. 13;

FIG. 16 is a top view of the wearable module showing the display element representing a low-intensity emotional state indicator;

FIG. 17 is a top view of the wearable module showing the display element representing a moderate-intensity emotional state indicator;

FIG. 18 is a top view of the wearable module showing the display element representing an elevated-intensity emotional state indicator compared to FIG. 16;

FIG. 19 is a top view of the wearable module showing the display element representing a high-intensity emotional state indicator and the maximum luminous intensity and color within the predetermined spectrum of colors;

FIG. 20 illustrates a plan view of the top surface of the wearable printed circuit board (PCB) with integrated physiological sensors;

FIG. 21 illustrates a plan view of the bottom surface of the wearable printed circuit board (PCB) detailing additional components including a microcontroller and a battery;

FIG. 22 illustrates a detailed schematic diagram of the sensor module components for acquiring physiological parameters;

FIG. 23 illustrates a detailed schematic diagram of the microcontroller unit (MCU) and related processing components, in accordance with one or more implementations; and

FIG. 24 illustrates a detailed schematic diagram of the power management integrated circuit (PMIC) and its associated power and communication interfaces.

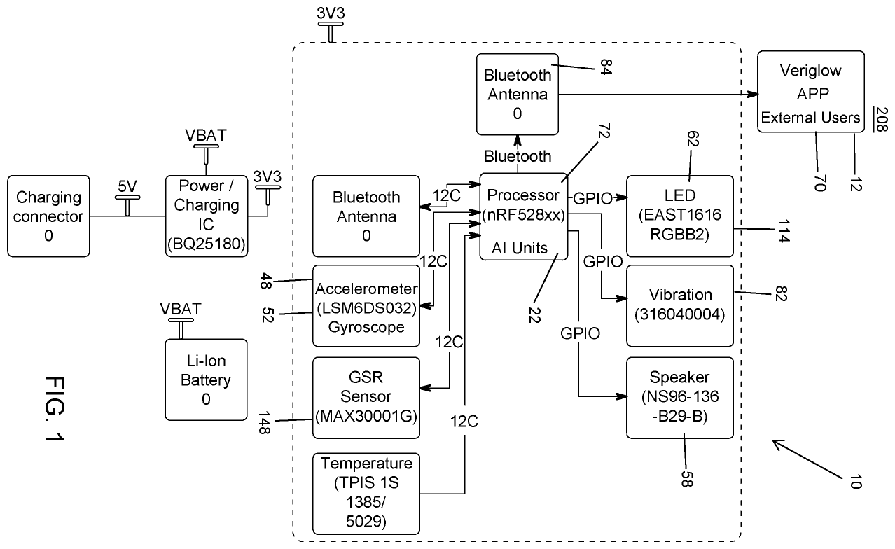

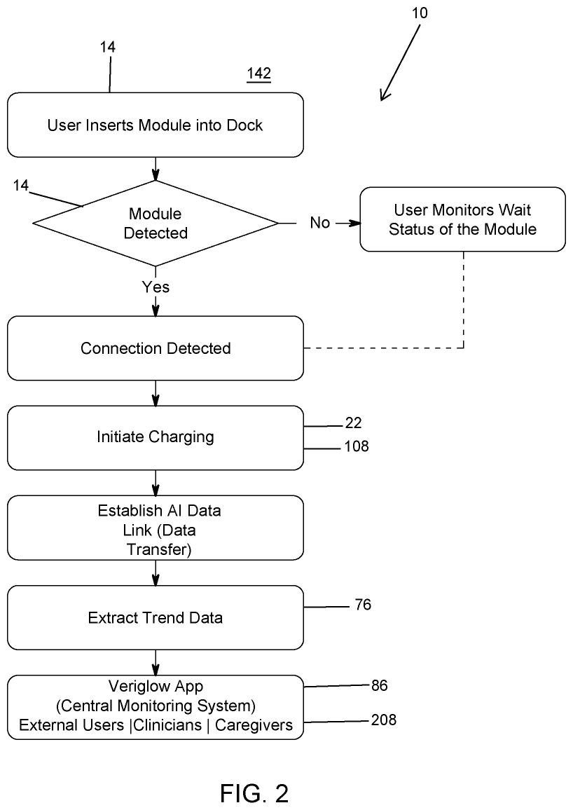

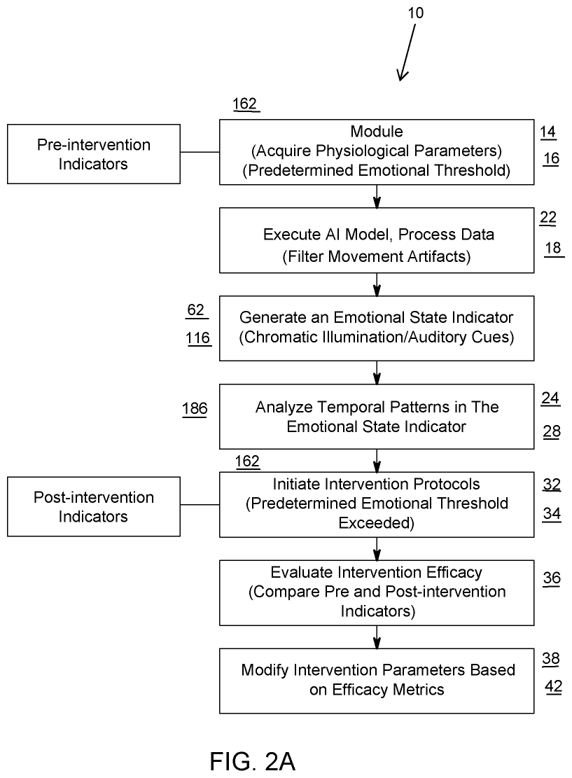

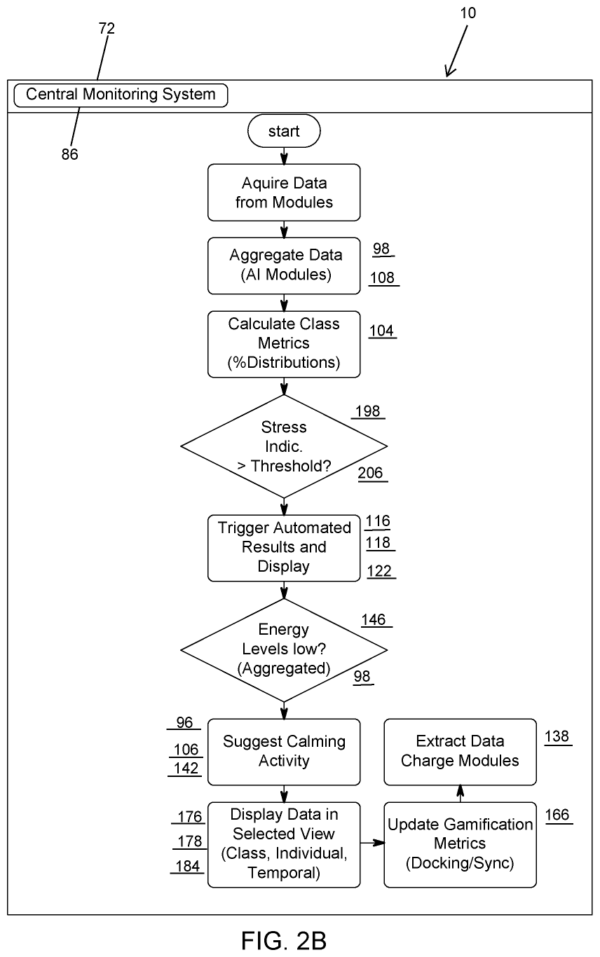

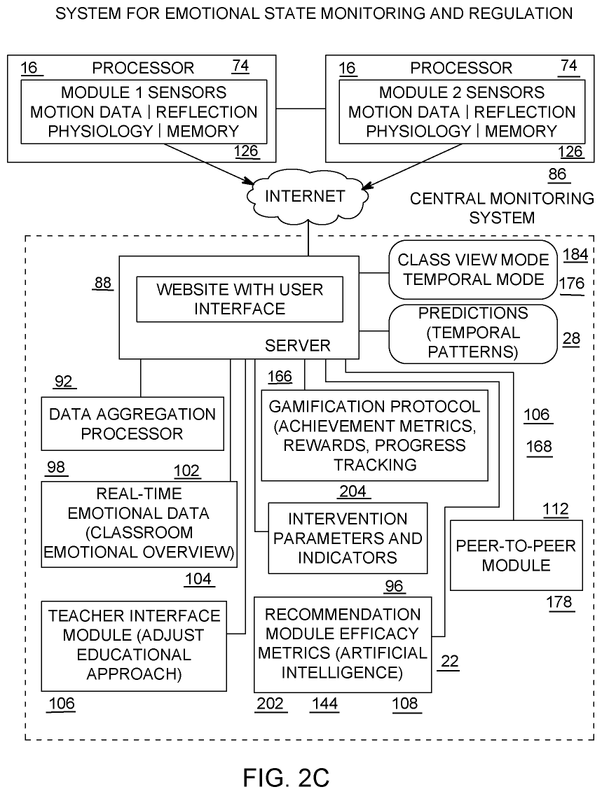

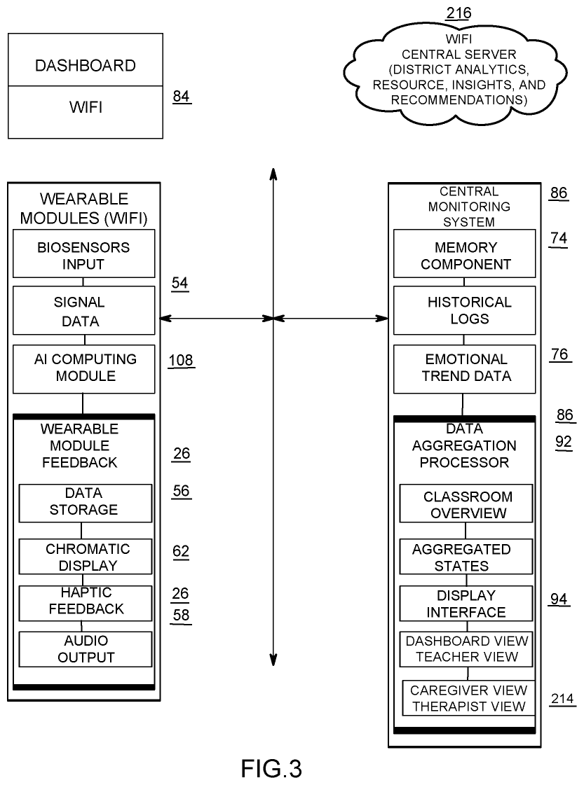

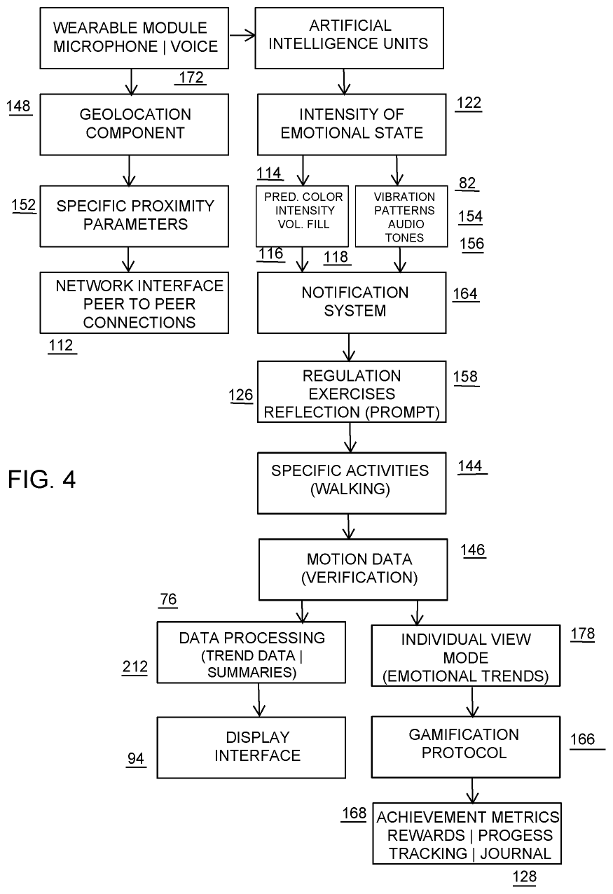

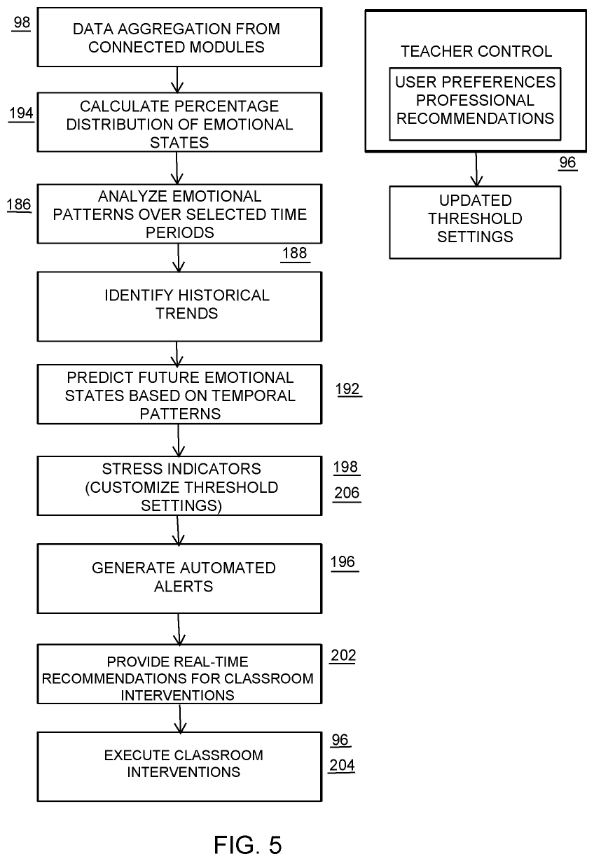

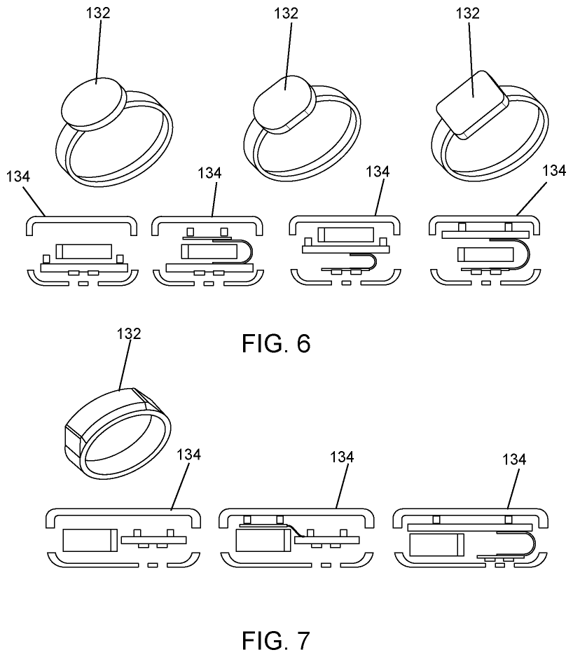

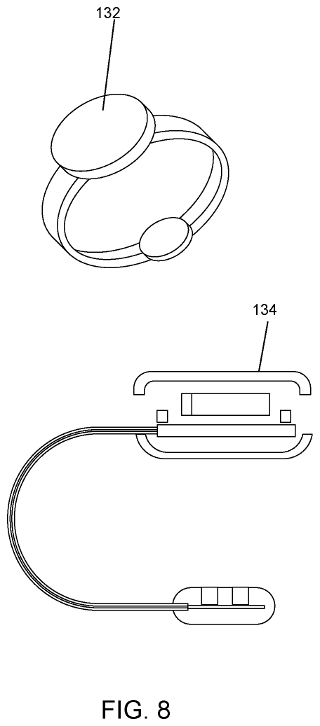

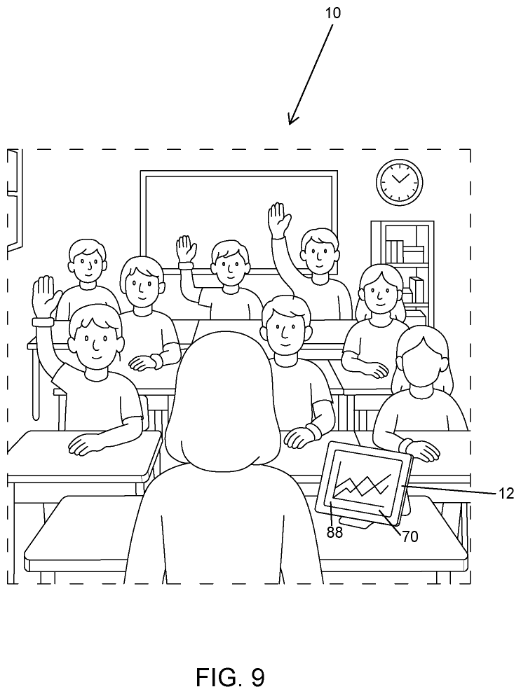

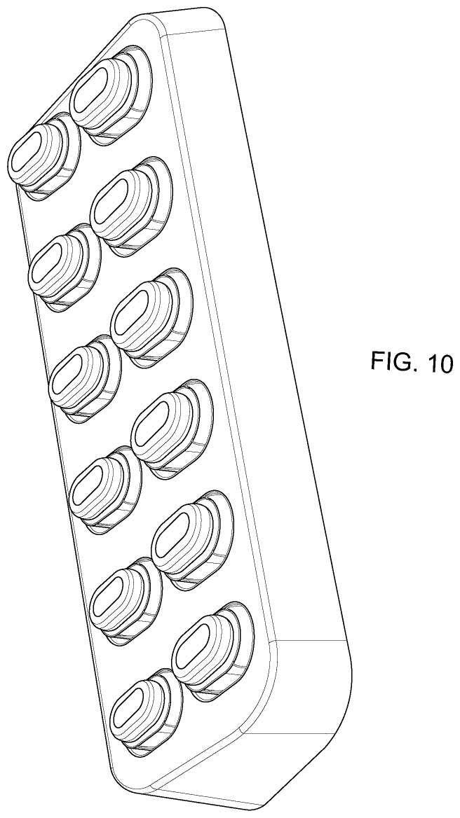

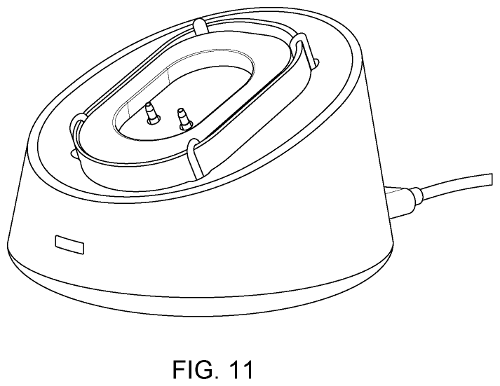

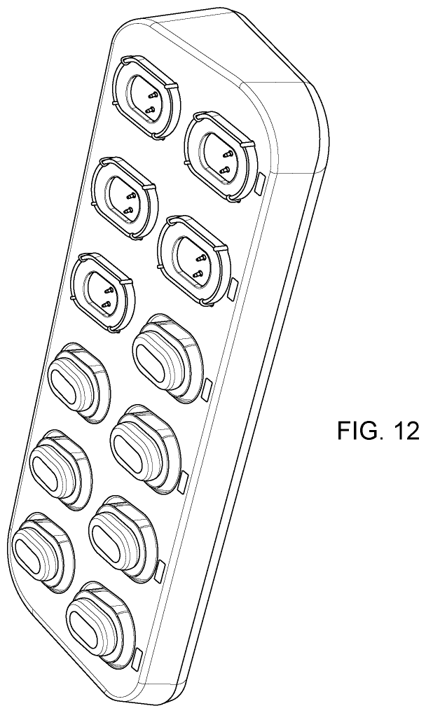

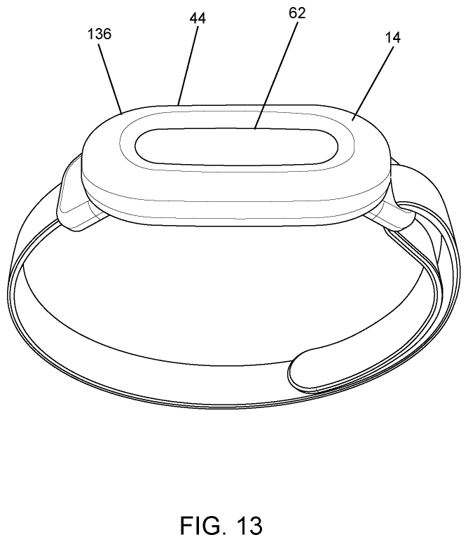

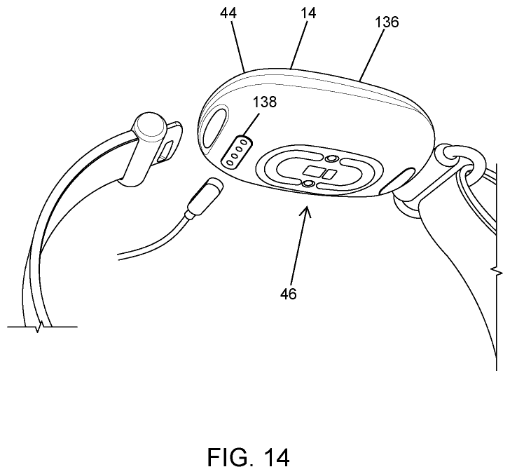

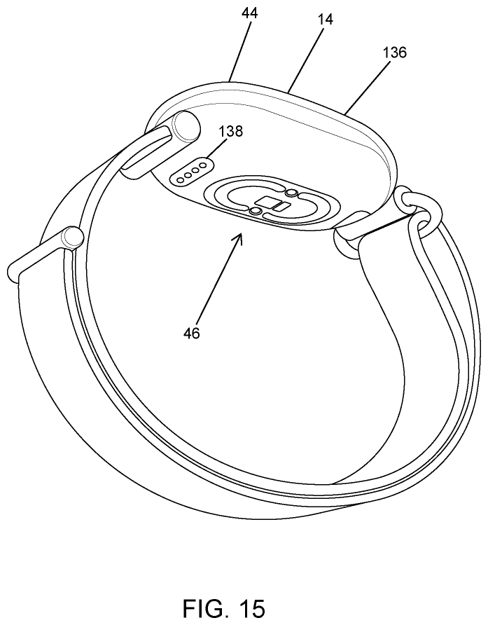

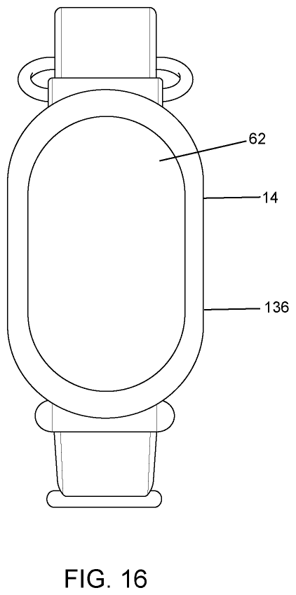

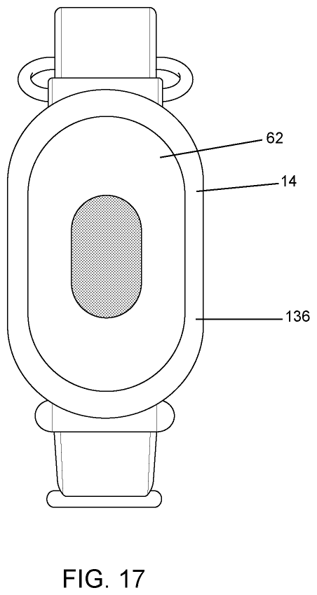

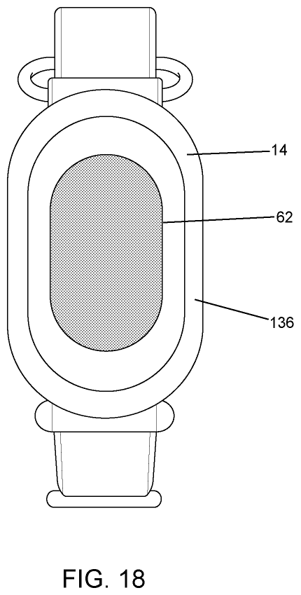

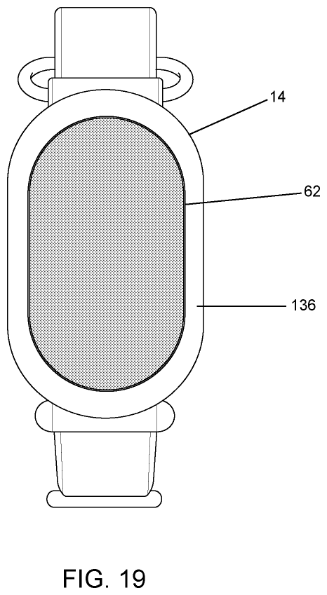

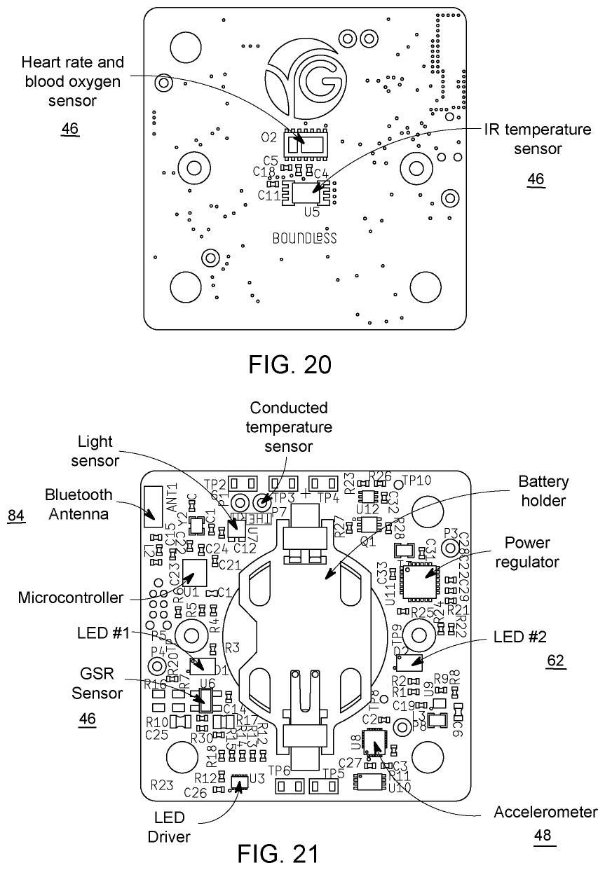

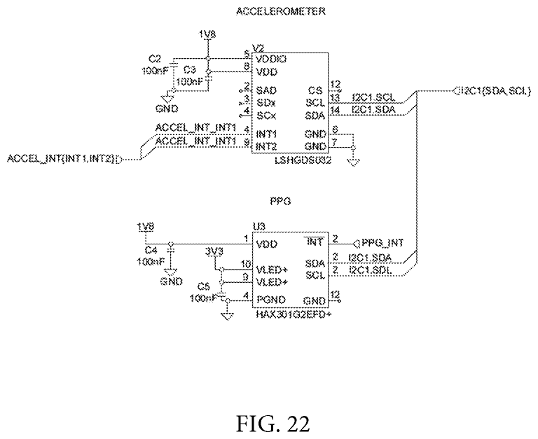

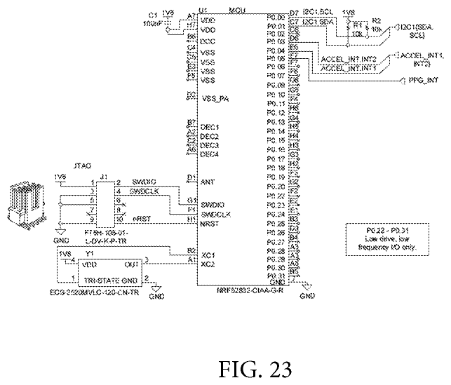

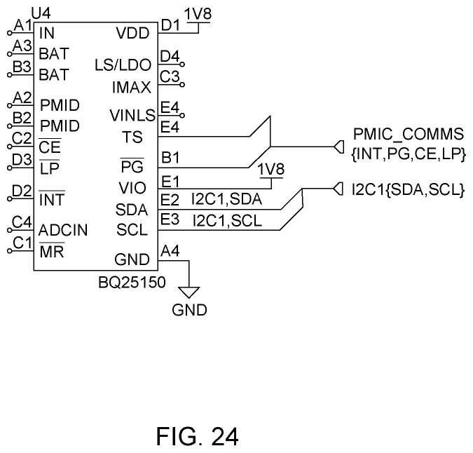

### DETAILED DESCRIPTION

The following detailed description provides a contemplated mode of carrying out a system and method for real-time emotional state monitoring and regulation 10, utilizing a wearable module 14 to assess physiological parameters 16 in educational, professional, clinical, and various other environments. Although the method is explained in relation to an illustrated embodiment focused on a classroom setting, it is understood that many possible modifications and variations can be made without departing from the spirit and scope of the disclosure. Therefore, the method 10 may be used by a plurality of individuals, including students, teachers, corporate employees, athletes, mental health professionals, parents, or caregivers, for monitoring emotional states in diverse settings such as offices, gyms, hospitals, or private homes. The purpose in all scenarios is to provide objective data and actionable insights that enhance focus, engagement, productivity, and overall emotional well-being as provided herein.

The system is designed to be highly versatile and capable of integrating a range of alternative physiological parameters 16 depending on the specific application requirements. While examples herein focus on electrodermal activity and cardiovascular data, the wearable module 14 may be configured to acquire other relevant biomarkers. Potential alternatives include measuring peripheral blood flow via photoplethysmography (PPG), muscle activity through electromyography (EMG), respiratory rate, blood oxygen saturation levels (SpO2), or even electroencephalogram (EEG) signals for more direct brain activity monitoring. The core system architecture is flexible and thereby allows for the substitution or addition of different biosensor arrays 46 to accommodate these various inputs and provide tailored emotional state monitoring and regulation across diverse fields.

The biofeedback mechanism 26 as described herein can be implemented through various alternative embodiments and thereby offering flexibility beyond the primary examples of chromatic illumination and auditory cues. The system is adaptable to utilize any suitable feedback mechanism capable of conveying emotional state indicators 24 to the user to facilitate self-regulation. Potential alternative biofeedback 26 methods may include haptic feedback, such as precisely modulated vibrations to represent varying emotional intensities or patterns 186. The system could also incorporate olfactory cues, releasing specific scents associated with calming or energizing states, or provide tactile feedback through changes in the surface texture or temperature of the wearable module 14. Furthermore, the biofeedback 26 could be entirely visual through a connected application interface, using complex graphical representations or data visualizations instead of color changes. These alternatives allow for customization based on user preference or clinical requirements and therefore expanding the scope of the invention to cover a broad range of sensory feedback mechanisms.

The intervention protocols 32 described herein encompass a broad spectrum of automated or suggested actions designed to mitigate undesirable emotional states or promote desired ones. While the present examples mention guided breathing exercises, physical activities, and meditation sequences, the system is not limited to these specific interventions. Alternative protocols can include, but are not limited to, generated music therapy playlists tailored to the user's emotional state, the presentation of cognitive behavioral therapy (CBT) prompts or journaling exercises through an associated application interface, or social interaction prompts suggesting connection with peers based on the peer-to-peer functionality described elsewhere. Furthermore, the system could be designed to initiate environmental changes through smart-home integration, such as adjusting room lighting, temperature, or activating aromatherapy. The core of the invention lies in the automatic initiation of any predefined or dynamically selected sequence of actions in response to exceeding an emotional threshold. This ensures a wide array of therapeutic or regulatory responses are possible.

The central monitoring system 86 is broadly conceived as an aggregation and analysis platform and its application can be expanded beyond educational environments. This apparatus 70 can be deployed in diverse settings, such as corporate wellness centers for employee stress monitoring, clinical research facilities for data collection, or even in home environments for family emotional health tracking. The system architecture is flexible, and its components can be varied. The communication interfaces may include wired connections or local area networks in addition to wireless. The data aggregation processor 72 could be a centralized cloud-based server, a local dedicated computer, or even distributed across peer-to-peer network nodes. Furthermore, the display interface 94 is not limited to typical visual monitors and could be a mobile application interface, an immersive virtual reality (VR) display for therapists, or an abstracted data feed for integration into existing enterprise software. Lastly, the recommendation module 96 can be adapted to suggest specific clinical interventions, team-building exercises, or personalized well-being resources. This thereby prevents a narrow interpretation limited only to classroom activity suggestions.

In one embodiment, the central monitoring system 86 is implemented, at least in part, as a software application executable on a user computing device, including but not limited to a smartphone, tablet, laptop, or web-based computing platform. The software application provides a display interface 94, communication interface 88, recommendation module 96, notification system 164, and user input mechanisms as described herein. The software application is configured to receive emotional state data from one or more wearable modules 14, present real-time and historical emotional state information 102, generate automated alerts 196, and initiate intervention protocols 32 based on predetermined or customized threshold 206.

The display interface 94 may provide role-based views, including but not limited to: (i) an educator view presenting aggregated emotional state distributions across a group without identifying individual users; (ii) an individual user view presenting personal emotional trends and biofeedback; (iii) a caregiver or therapist view presenting authorized individual emotional trend data; and (iv) an administrative or district-level view presenting anonymized analytics and longitudinal trends across multiple environments.

### Digital Ecosystem and Peer Support Embodiment

In certain embodiments, the system can further comprise a networked digital platform accessible via mobile application and/or web interface configured to facilitate user interaction within an emotionally adaptive support ecosystem. Biometric data collected from wearable modules 14 may be processed to generate anonymized emotional state indicators 24, which are used by the system to algorithmically identify similarity profiles among users based on emotional patterns, behavioral trends, temporal proximity, and contextual factors.

The system may generate dynamic peer clusters or matched pairings composed of users exhibiting substantially similar emotional state trajectories. Matching logic may incorporate similarity scoring models that evaluate emotional intensity, duration, recurrence patterns, and longitudinal modeling data.

Users may communicate within the ecosystem through one or more modalities including, but not limited to, voice-based communication sessions, text-based messaging, moderated group discussions, asynchronous support threads, or guided intervention rooms. In certain embodiments, user identity may remain anonymized within peer environments.

The digital ecosystem may further include AI-assisted moderation systems configured to detect escalation patterns, prevent reinforcement loops, limit unhealthy dependency behaviors, and escalate concerns to designated human supervisors when predefined intervention criteria are met.

In some embodiments, the peer ecosystem operates in conjunction with institutional oversight frameworks, allowing authorized personnel to configure participation parameters, visibility permissions, and safety protocols in accordance with organizational policy.

The platform may operate as part of a hybrid on-device and cloud-based architecture, wherein biometric data is processed locally and/or remotely to facilitate adaptive matching and support coordination.

The sensor array 46, as described herein, is a versatile component within the wearable module 14, but its configuration and capabilities are not limited to solely monitoring heart rate, skin temperature, and body motion. The inventors contemplate a broad range of sensor integrations designed to monitor various physiological parameters 16 indicative of emotional states. Alternative sensor technologies may be incorporated, such as those configured to detect blood pressure continuously, monitor respiratory patterns through impedance pneumography, or measure blood oxygen saturation (SpO2) via pulse oximetry. The physical integration of these sensor elements can also vary, potentially being integrated directly into other materials rather than strictly within the wearable module 14 itself. The core concept encompasses any sensor configuration that provides data relevant to a user's physiological state for the purpose of emotional monitoring and regulation, thereby avoiding a restrictive interpretation of the specific sensors listed.

The recommendation module 96 described herein is a versatile component of the central monitoring system, and its functionality extends well beyond merely suggesting classroom activities based on aggregated emotional states 98. This module can be configured to operate in various contexts, such as recommending specific team-building exercises in a corporate setting, suggesting tailored therapeutic regimens in a clinical environment, or proposing personalized lifestyle adjustments for an individual user in a home setting. The nature of the suggestions can also be diversified to include outputting nutritional advice based on stress levels, recommending specific mindfulness applications or guided meditations, and initiating environmental controls (e.g., smart lighting or audio systems). The core function remains the intelligent generation of actionable advice based on processed emotional state data. This leverages a variety of output mechanisms and adapting to diverse application domains far beyond the specific examples provided in the claims.

The physical docking interface 142 described herein represents one embodiment of how the system interacts with the wearable modules 14; however, the scope is intended to cover various methods for both power transfer and data management. The invention is not limited to a singular physical connection mechanism. Alternative methods for charging could include wireless charging that could eliminate the need for direct metal-to-metal contact. Similarly, data extraction is not restricted to occurring only while physically docked. The system could utilize wireless communication protocols such as Bluetooth low energy or Wi-Fi to extract stored data automatically and continuously to the central system or to the mobile application. Charging and data extraction could also be implemented via entirely separate methods. The core concept is any system or combination of components that facilitates the necessary power management and data synchronization to avoid a narrow interpretation limited only to a specific physical docking port 142.

While the present disclosure provides a detailed description of the emotional monitoring and regulation system utilizing a specific gamification protocol embodiment 166 (e.g., comprising achievement metrics 168, rewards for emotional regulation improvements, and progress tracking goals), it is to be understood that this specific implementation is illustrative and not restrictive. The invention contemplates numerous modifications, alternative constructions, and equivalents. The described gamification elements 166 are examples of the principles of the invention and may be varied, combined, or omitted, provided they achieve similar functional results within the scope of the appended claims. Therefore, the invention is not limited to the specific protocol defined herein but encompasses various alternative systems and methods for engaging users in emotional self-awareness and regulation activities.

### Definitions

As used herein, the term “comprising” means various components can be conjointly employed in the system and method for real-time emotional state monitoring and regulation 10 of the present invention. Accordingly, the term “comprising” encompasses the more restrictive terms “consisting essentially of” and “consisting of”;
Wearable module 14 as described herein is a device comprising a detachable housing 44 that contains biosensor arrays 46, an accelerometer 48, a gyroscope 52, signal processing, data storage, haptic feedback, audio output, and chromatic display elements, adaptable for wear as a ring, bracelet, necklace, earring, or the same;
Physiological parameters 16 as described herein are data acquired by the wearable module 14, specifically dermal temperature, electrodermal activity, cardiovascular waveforms, and motion data 146;
Emotional state indicator 24 as described herein is a generated output based on processed physiological parameters 16 that represents a user's emotional state;
Biofeedback 26 as described herein is a system of variable chromatic illumination and auditory cues provided by the module corresponding to the emotional state indicator 24;
Intervention protocols 32 as described herein is a redefined actions or sequences (e.g., guided breathing exercises, physical activities, meditation) initiated when emotional thresholds are exceeded;
Central Monitoring System 86 as described herein is an apparatus 70 used in educational environments comprising communication interfaces, a data aggregation processor 72, a display interface 94, and a recommendation module 96 configured to receive data from multiple wearable modules 14. The data aggregation processor that performs statistical analysis on the concurrent data streams originating from the plurality of wearable modules. Specifically, the system is configured to calculate percentage distributions, which represent the ratio of specific emotional classifications (e.g., ‘focused,’ ‘anxious,’ or ‘calm’) relative to the total number of active users in the classroom environment. This provides the educator with a quantified overview of the collective classroom climate at any given moment;
Sensor array 46 as described herein is a component within the wearable module 14 configured to monitor physiological parameters 16 including heart rate, skin temperature, and body motion;
Recommendation module 96 as described herein is a component of the central monitoring system 86 configured to suggest specific classroom activities (e.g., energizing or calming exercises, specific breathing exercises, environmental adjustments including lighting or sound changes, 5-minute breaks or the same to transition to different educational activities designed to return the classroom to an optimal emotional state for learning) based on aggregated emotional states 98;
Physical docking interface 142 as used herein is a component used by the central monitoring system 86 configured to charge 138 wearable modules 14 and extract stored data. Each wearable module 14 includes a dedicated charge port 138 or electrical contact interface configured to mate with the physical docking interface 142 of the central monitoring system. This physical connection establishes a simultaneous power and data link, enabling the central monitoring system 186 to replenish the battery of the wearable module 14 while concurrently initiating a high-speed data transfer to extract emotional trend data 76 from the memory component 74; and
Gamification protocol 166 as used herein is a system implemented by the central monitoring system 86 that includes achievement metrics 168, rewards for emotional regulation improvements, and progress tracking goals.

Therefore, a method for emotional state monitoring and regulation 10, implemented on a computer system 12, is described herein. The process begins with a wearable module 14 acquiring various physiological parameters 16 from a user, such as dermal temperature, electrodermal activity, cardiovascular waveforms, and motion data 146. Any acquired cardiovascular waveforms and motion data 146 are then processed to filter out movement artifacts 18, ensuring accurate data analysis. Based on these refined parameters, an emotional state indicator 24 is generated.

In the context of biometric sensing, movement artifacts 18 refer to signal distortions and transient noise introduced into the physiological data stream caused by the physical displacement of the wearable module 14 relative to the user's skin. These artifacts 18 typically manifest as high-amplitude spikes, baseline wanders, or erratic oscillations that can mask or mimic legitimate biological signals, such as the subtle fluctuations in electrodermal activity or the rhythmic peaks of a cardiovascular waveform. In the present invention, these artifacts 18 are primarily generated by the kinetic energy of the user's limbs, adjustments of the detachable housing, or changes in sensor-to-skin contact pressure during physical activity. By utilizing concurrent data from the accelerometer 48 and gyroscope 52, the system identifies these non-biological signals in real time, allowing the processing unit to apply digital filters or adaptive cancellation algorithms to isolate the clean physiological data required for accurate emotional state classification.

The system provides biofeedback 26 to the user through variable chromatic illumination and auditory cues that correspond to this emotional state indicator 24. The method further involves analyzing temporal patterns 28 within the emotional state indicator 24. Intervention protocols 32 are initiated when predetermined emotional thresholds 34 are exceeded. The efficacy of these interventions is then evaluated through a comparative analysis of indicators before and after the intervention, allowing for the modification of intervention parameters 38 based on the resulting efficacy metrics 42.

The wearable module 14 comprises a detachable housing 44 designed to be highly versatile. This housing contains essential components, including biosensor arrays 46, an accelerometer 48, a gyroscope 52, signal processing components 54, data storage elements 56, haptic feedback mechanisms 58, an audio output component 58, and chromatic display elements 62. Through modular attachment mechanisms 134, this detachable housing, which comprises a rounded, pebble-shaped enclosure 136, can be adapted for wear as a ring, bracelet, necklace, or earring.

A system for emotional state monitoring 10 in educational environments as provided herein further incorporates a plurality of wearable modules 14 and a central monitoring system. Each wearable module 14 is equipped with a sensor array 46 configured to monitor physiological parameters 16 like heart rate, skin temperature, and body motion. A processor 72 within each module is configured to analyze these parameters in real-time, and a memory 74 component stores emotional trend data 76. Visual feedback is provided by a chromatic display element configured to represent a user's emotional state through color variations, while a vibration mechanism 82 provides tactile feedback for emotional regulation. Each module also includes a wireless communication transceiver 84.

The central monitoring system 86 is the hub of the apparatus 70. It comprises a communication interface 88 to receive data from all modules, a data aggregation processor 92 configured to compile emotional state data, a display interface 94 to present individual and aggregated emotional states 98 visually, and a recommendation module 96 configured to suggest classroom activities based on the aggregated emotional states 98. The system is specifically configured to display real-time emotional data 102 when a module is connected, generate classroom-wide emotional overviews 104, and enable teachers to adjust educational approaches 106 based on aggregated emotional states 98. The display interface 94 offers a class view mode 176 for aggregate metrics, an individual view mode 178 for single-user trends, and a temporal view mode 184 for displaying patterns 186 over time 188. The central system calculates percentage distributions 194 of emotional states, generates automated alerts 196 for stress thresholds, and provides real-time recommendations 202 for classroom interventions 204. To further enhance the utility of the disclosure provided herein, aggregated emotional data states 98 are analyzed to generate district-level insights and resource recommendations 216. The aggregated emotional data 98 can be further collected across multiple classrooms or similar facilities.

Further functionality of the central system includes charging the wearable modules 14 via a physical docking interface 142 to extract stored emotional trend data 76. A gamification protocol 166 is implemented, comprising achievement metrics 168 for consistent docking or synchronization, rewards for emotional regulation improvements, and progress tracking for emotional self-awareness goals. The recommendation module 96 suggests specific activities 144, such as energizing exercises for low energy levels or calming exercises for elevated stress levels, utilizing motion data 146 from the modules to verify participation in those exercises.

Additional method refinements include networking features, such as establishing peer-to-peer connections between users experiencing similar emotional states via a networking interface 112. Determining user proximity through a geolocation component 148 allows connections within specified proximity parameters 152. Biofeedback 26 can be refined by transitioning chromatic illumination through a spectrum of colors and modulating luminous intensity 116 and volumetric fill 118 to represent emotional intensity 122.

Distinct vibration patterns 154 and audio tones 156 corresponding to different emotional states can also be generated. These states are determined locally by an artificial intelligence processing unit 22 integrated within the wearable module 14, which executes neural network models to perform real-time classification of physiological data. By utilizing this on-device artificial intelligence processing 22, the system achieves low-latency inference and enhanced data privacy through an edge-computing architecture. Adaptation and customization are further facilitated by employing machine learning algorithms to adapt intervention protocols 32 based on historical efficacy data and allowing customization of threshold settings 206 based on user preferences. For example, a user may manually adjust sensitivity thresholds to prevent “false positive” alerts during high-intensity exercise, or a teacher may lower a stress-trigger threshold for a student known to require earlier intervention. Furthermore, these thresholds 206 may be customized to account for different baseline physiological ranges, such as a naturally high resting heart rate or varying levels of skin conductance. The artificial intelligence processing 22 unit may further utilize transfer learning to refine these settings by adapting generalized emotional models to the unique physiological baseline of an individual user.

In one or more embodiments, as specified in Claim 4, the artificial intelligence processing unit 22 is implemented as a specialized edge-computing module 108 integrated directly within the wearable housing. The module 108 transforms raw physiological sensor data into emotional state classifications locally, without requiring a constant uplink to a remote server. This configuration allows for the near-instantaneous provision of biofeedback 26 (e.g., chromatic illumination).

The artificial intelligence processing unit 22 may utilize transfer learning protocols. In this context, the module 14 is pre-loaded with a generalized emotional model trained on large-scale datasets of diverse users. Once deployed, the edge-computing module 108 monitors the specific user's physiological baseline (e.g., resting heart rate or typical skin conductance levels) and applies a transfer learning layer to fine-tune the model weights. This adaptation allows the module 14 to recognize emotional shifts relative to that specific user's unique baseline rather than a generic average. This increases the accuracy of the emotional state indicator 24 while maintaining the efficiency of artificial intelligence processing.

Intervention protocols 32 themselves can include guided breathing exercises, physical activities, 5-minutes breaks, or meditation sequences guided by the illumination and cues. Emotional regulation exercises 158 can be initiated at predetermined intervals 162 through a notification system 164. The method supports user reflection 126, where users are prompted for reflection 126 based on the indicator, and temporal emotional state data is stored in a digital journal 128, potentially including voice annotations 172 recorded via a microphone 174 integrated into the wearable module 14. The system is also capable of predicting future emotional states 192 based on identified temporal patterns 28. Authorized caregivers are therefore provided limited access to emotional trend summaries 212 via a caregiver interface and a therapist dashboard can be provided wherein licensed clinicians 208 are provided access to the emotional trend data and the intervention parameters for therapeutic oversight. Wherein the therapeutic oversight an include additional supervision, data review, or the same for monitoring a user.

### Acoustic Input

The wearable module 14 can further comprise an integrated microphone 174 configured to capture acoustic input that allows the user to record voice annotations as a verbal reflection of their perceived emotional state. These voice annotations can be timestamped and stored as part of the real-time emotional state data, creating a multi-modal record that pairs objective physiological metrics with subjective user insights. This audio data may be processed via speech-to-text algorithms or stored as compressed audio files within the digital journal 128 to provide context for detected emotional shifts during subsequent review by the user or an educator.

### Mapping of System Outputs Biofeedback

Table 1, presented below, illustrates an exemplary mapping of system outputs (biofeedback 26) to specific emotional state indicators 24 and corresponding intervention thresholds, which can be customized based on user preferences or professional recommendations as described in Claim 21. This data mapping resides within the system memory 74 for reference during operation.

TABLE 1
Emotional Indicator to Biofeedback Mapping

| Emotional State | Chromatic | | | |
|---|---|---|---|---|
| Indicator | Illumination | | Vibration | Intervention |
| (Intensity Level) | (Color/Intensity) | Auditory Cue | Pattern | Threshold |
| Low | Soft Blue (Low | Gentle hum | None | N/A |
| Stress/Calm | Luminous | (Low Tone) | | |
| | Intensity) | | | |
| Neutral/Engaged | Green (Medium | N/A | N/A | N/A |
| | Intensity) | | | |
| Elevated | Yellow/Orange | Ascending | Single pulse | Threshold 1 |
| Stress/Anxiety | (High Intensity, | chime | | (Customizable) |
| | modulating | | | |
| | volumetric fill | | | |
| | level) | | | |
| High | Red/Flashing | Rapid beeps | Rapid pulses | Threshold 2 |
| Alert/Distress | (Very High | (High Tone) | (Alert pattern) | (Customizable) |
| | Intensity, | | | |
| | maximum fill | | | |
| | level) | | | |

This table defines the “variable chromatic illumination” and “auditory cues” described in Claims 6 and 18 respectively. For instance, a “Low Stress/Calm” indicator can correspond to a soft blue illumination with low luminous intensity 116, a gentle low-tone hum, and no vibration. For example, and without limitations, a high alert/stress indicator 198 can map to rapid-flashing red light at maximum intensity, rapid high-tone beeps, and rapid pulse vibrations which are initiated upon exceeding a customizable threshold 2. This exemplary structured mapping would allow the system to provide appropriate biofeedback 26 and initiate specific intervention protocols 32 tailored to the detected emotional state of a user.

### Examples of Efficacy Evaluation

Table 2, presented below, provides exemplary efficacy metrics 42 for emotional regulation interventions in accordance with the disclosed apparatus 70 and methods. Specifically, Table 2 illustrates the “evaluating intervention efficacy” and “modifying intervention parameters” steps of Claim 1. As demonstrated by user A's data, the system can be shown to successfully reduce GSR activity (stress level) from a pre-intervention value of 7.2 μS to a post-intervention value of 4.5 μS, representing a significant-37.5% change and an assessment of “highly efficacious”. This efficacy evaluation subsequently informed the “Intervention Parameter Modification” column, leading to the adjustment: “Biofeedback duration increased by 10% for future similar events”. Similarly, user B's heart rate (HR) was reduced from 98 BPM (elevated) to 75 BPM (normal range). This demonstrates a −23.5% efficacy. The data f for user C, showing a minimal +2.0% change in dermal temperature and “minimally efficacious” assessment, can further support the system's ability to adapt by triggering a parameter modification for “Chromatic illumination spectrum narrowed to calming blue/green range.” Therefore, this exemplary table provides specific data points that can validate the effectiveness and dynamic adaptability of the claimed invention in real-world scenarios.

TABLE 2
Prophetic Efficacy Metrics for Emotional Regulation Interventions

| | Pre- | Post- | | | |
|---|---|---|---|---|---|
| | Intervention | Intervention | | | |
| | Indicator | Indicator | | | |
| | Value | Value | Percentage | | Intervention |
| | (Avg. | (Avg. | Change | Efficacy | Parameter |
| Metric | Score) | Score) | (%) | Assessment | Modification |
| User A: | 7.2 μS | 4.5 μS | −37.5% | Highly | Biofeedback |
| GSR | (High | (Moderate | | Efficacious | duration |
| Activity | Stress) | Stress) | | | increased by |
| (Stress | | | | | 10% for future |
| Level) | | | | | similar events |
| User B: | 98 BPM | 75 BPM | −23.5% | Efficacious | Auditory cue |
| Heart Rate | (Elevated) | (Normal | | | volume |
| (HR) | | Range) | | | modulated for |
| (BPM) | | | | | optimal comfort |
| | | | | | level |
| User C: | 35.1 | 35.8 | +2.0% | Minimally | Chromatic |
| Dermal | | | | Efficacious | illumination |
| Temp | | | | | spectrum |
| (Degrees | | | | | narrowed to |
| Celcius) | | | | | calming |
| | | | | | blue/green range |
| Classroom | 6.5 | 6.4 | −1.5% | Ineffective | Recommendation |
| Aggregate | (Moderate) | (Moderate) | | (Low | module suggests |
| (Avg. | | | | Energy) | “energizing |
| Stress) | | | | | exercises” via |
| | | | | | app interface |

### Privacy and Access Control

In certain embodiments, the system can implement privacy controls and permission logic configured to restrict access to emotional state data based on user roles, institutional policies, and applicable privacy regulations. Access permissions may be dynamically assigned to external users 208 such as educators, administrators, caregivers, clinicians, or individual users, such that only authorized aggregated or anonymized data is displayed where appropriate.

### Design

The physical design of the module 14 can vary significantly while maintaining its core functionality. The module housing could be any ergonomic shape, not limited to the rounded, pebble-shaped enclosure 136 mentioned in the claims. Possible variations include square, rectangular, or more organic, aesthetic forms, depending on the target user population (e.g., sleek, minimalist designs for adults; colorful, playful designs for children). The module's placement on the body is also flexible; while claims mention a ring, bracelet, necklace, or earring through modular attachment mechanisms 134, the module 14 could also be incorporated into clothing (e.g., integrated into a shirt collar or arm band), attached via an adhesive patch, or integrated into a watch strap.

### Materials

The types of materials used for the module's construction can be chosen based on comfort, durability, and biocompatibility. Common materials for the housing and straps include medical-grade silicone, various flexible polymers like polyurethane (PU) or thermoplastic elastomers (TPEs), rigid plastics like polyethylene, or metals such as stainless steel for structural components or sensors. The materials should be durable enough to withstand daily wear, resistant to sweat and UV exposure, and hypoallergenic for prolonged skin contact.

### Manufacturing

The manufacturing of the electronic components and final assembly for the wearable module 14 and central monitoring system 86 can be achieved through various established production methods suitable for consumer electronics. These methods may involve standard printed circuit board assembly (PCBA) processes, injection molding for the various housing components (e.g., the rounded, pebble-shaped enclosure), and automated assembly lines. The choice of specific manufacturing techniques and locations will be determined by factors such as the required production volume, desired quality control standards, and overall supply chain logistics to ensure efficient production.

### Cost Considerations

Cost considerations are an inherent part of the manufacturing process. The total cost of production, including the materials for the Bill of Materials (BOM) and the associated assembly labor, can vary depending on the scale of manufacturing and the specific components selected. The ratio of material costs to total manufacturing costs is a common metric in the industry, and the objective is typically to achieve a cost structure that supports a viable commercial product while meeting all performance and durability requirements for use in various environments, including educational and clinical settings.

Certain components necessary to the operation of this disclosure are not shown or described in detail because they are components well known to those in the relevant arts. These components can include requisite modules, dashboards, devices, databases, and similar components.

While the invention has been described in detail and with reference to specific embodiments thereof, it will be apparent to one skilled in the art that various changes and modifications can be made therein without departing from the spirit and scope thereof.

What is claimed is:

1. A method for emotional state monitoring and regulation, implemented on a computer system, comprising:
acquiring, by a wearable module, physiological parameters such as dermal temperature, electrodermal activity, cardiovascular waveforms, and motion data;
processing the physiological parameters to filter movement artifacts in real time;
generating, via an artificial intelligence processing unit, an emotional state indicator based on the physiological parameters;
providing biofeedback through variable chromatic illumination and auditory cues corresponding to the emotional state indicator;
analyzing temporal patterns in the emotional state indicator; initiating intervention protocols when predetermined emotional thresholds are exceeded;
evaluating intervention efficacy through comparative analysis of pre- and post-intervention indicators; and
modifying intervention parameters based on efficacy metrics.

2. The method of claim 1, wherein the wearable module comprises a detachable housing containing biosensor arrays, an accelerometer, a gyroscope, signal processing components, data storage elements, haptic feedback mechanisms, an audio output component, and chromatic display elements.

3. An apparatus for emotional state monitoring in educational environments, comprising:
a plurality of wearable modules, each wearable module comprising:
a sensor array configured to monitor physiological parameters including heart rate, skin temperature, and body motion;
an artificial intelligence processing unit configured to execute real-time emotional state classification;
a processor configured to analyze the physiological parameters in real-time;
a memory component configured to store emotional trend data;
a chromatic display element configured to visually represent a user's emotional state through color variations;
a vibration mechanism configured to provide tactile feedback for emotional regulation; and
a wireless communication transceiver;
a central monitoring system comprising:
a communication interface configured to receive emotional state data from the plurality of wearable modules;
a data aggregation processor configured to compile the emotional state data from multiple wearable modules;
a display interface configured to present individual and aggregated emotional states through visual representations; and
a recommendation module configured to suggest classroom activities based on the aggregated emotional states;
wherein the central monitoring system is configured to:
display real-time emotional state data when a wearable module is connected;
generate classroom-wide emotional overviews; and
enable teachers to adjust educational approaches based on the aggregated emotional states.

4. The apparatus of claim 3, wherein the artificial intelligence processing unit comprises an edge-computing module configured to perform on-device inference of emotional states, thereby reducing latency and increasing data privacy.

5. The method of claim 1, further comprising establishing, via a networking interface, peer-to-peer connections between users experiencing similar emotional states based on their respective emotional state indicators.

6. The method of claim 1, wherein providing biofeedback comprises transitioning the chromatic illumination through a predetermined spectrum of colors corresponding to varying intensities of the emotional state indicator and modulating a luminous intensity and a volumetric fill level of the chromatic illumination to represent an intensity of the emotional state.

7. The method of claim 1, further comprising employing the artificial intelligence processing unit to adapt intervention protocols based on efficacy metrics.

8. The method of claim 1, further comprising: prompting user reflection based on the emotional state indicator; and storing real-time emotional state data in a digital journal.

9. The method of claim 1, wherein authorized caregivers are provided limited access to emotional trend summaries via a caregiver interface.

10. The method of claim 1, wherein external users such as licensed clinicians are provided access to the emotional trend data and the intervention parameters for therapeutic oversight.

11. The method of claim 2, further comprising adapting the detachable housing for wear as a personal adornment through modular attachment mechanisms, wherein the detachable housing comprises a rounded, pebble-shaped enclosure.

12. The apparatus of claim 3, wherein the central monitoring system is configured to charge the wearable modules via a physical docking interface while extracting the emotional trend data from the memory component of at least one of the plurality of wearable modules.

13. The apparatus of claim 3, wherein the recommendation module is configured to suggest specific activities, utilizing motion data from the wearable modules to verify participation in the specific activities.

14. The method of claim 5, further comprising determining user proximity through a geolocation component for connecting users within specified proximity parameters.

15. The method of claim 1, further comprising generating distinct vibration patterns and distinct audio tones corresponding to different emotional state indicators.

16. The method of claim 1, further comprising initiating emotional regulation exercises at predetermined intervals through a notification system.

17. The apparatus of claim 3, wherein the central monitoring system implements a gamification protocol comprising: achievement metrics for consistent wearable module docking or synchronization; rewards for emotional regulation improvements; and progress tracking for emotional self-awareness goals.

18. The method of claim 1, wherein the intervention protocols are guided by the chromatic illumination and the auditory cues.

19. The method of claim 8, where real-time emotional state data comprises recording voice annotations of emotional state reflections via a microphone integrated into the wearable module.

20. The apparatus of claim 3, wherein the display interface comprises: a class view mode displaying the aggregated emotional states; an individual view mode displaying emotional trends of a single wearable module which corresponds to the emotional trends of a single-user; and a temporal view mode displaying emotional patterns over selected time periods.

21. The method of claim 1, further comprising predicting future emotional states based on identified temporal patterns.

22. The apparatus of claim 3, wherein the central monitoring system is configured to: calculate percentage distributions of emotional states across connected wearable modules; generate automated alerts when stress indicators exceed the predetermined emotional thresholds; and provide real-time recommendations for classroom interventions.

23. The apparatus of claim 3, wherein the aggregated emotional states are analyzed to generate district-level insights and resource recommendations.

24. The method of claim 1, further comprising customizing threshold settings for initiating the intervention protocols.
